(19) United States
(12) Patent Application Publication
(10) Pub. No.: US 2004/0116341 A1
(43) Pub. Date: Jun. 17, 2004

(54) Methods for ameliorating childhood infections

(76) Inventor: Michael J. Holtzman, St. Louise, MO (US)

Correspondence Address: Kate H Murashige, Morrison & Foerster, Suite 500, 3811 Valley Centre, San Diego, CA, 92130, US

(21) Appl. No.: 10/416,938

(22) PCT Filed: Oct. 23, 2001
(86) PCT No.: PCT/US01/45244

(51) Int. Cl. A61K 38/17

(52) U.S. Cl. 514/12

(57) ABSTRACT

Methods to treat paramyxovirus infection are disclosed, which comprise administering RANTES protein or an expression system therefor. Also disclosed are methods to identify individuals susceptible to paramyxovirus infection.

### TECHNICAL FIELD

The invention is directed to the use of RANTES and its analogs in the prevention and treatment of infections caused by paramyxovirus. More specifically, the invention concerns both treatment and diagnosis of paramyxoviral infection, especially in children.

### BACKGROUND ART

In infants and young children, the most common cause of serious respiratory disease is paramyxoviral infection. Not only does the virus cause immediate troublesome respiratory conditions, it also predisposes the subject to the development of asthma. An effective means to prevent or ameliorate this infection would be a significant advance in public health. In addition, a screening test to identify those subjects who are susceptible to these infections would have a beneficial effect in permitting precautionary steps to be taken, including those described below. The present invention identifies an endogenous agent useful in combating this infection. As the agent is endogenous, not only does the endogenous agent provide a treatment means for this condition, it also provides a method to screen for susceptibility.

The present invention is based on the discovery that the RANTES protein is this endogenous agent.

RANTES is a 68 amino acid protein which is a “C-C” chemokine; other members of this family include MCP-1 and MCP-3. C-C chemokines (so designated based on the relative positions of the first cysteine residues to occur in the sequence) are generally active on a variety of leukocytes including monocytes, lymphocytes, eosinophils, basophils, NK cells and dendritic cells. There is an extensive literature describing this secreted protein as well as its analogs. For example, Struyf, S., et al., Eur. J. Immunol. (1998) 28:1262-1271 isolated a natural form of human RANTES lacking two N-terminal residues (RANTES (3-68)) from stimulated sarcoma cells, fibroblasts and leukocytes and report that this form showed more than ten-fold reduction in chemotactic potency for monocytes and eosinophils. RANTES (3-68) inhibited RANTES (1-68) induced chemotaxis as well as chemotaxis induced by related cytokines.

Another RANTES antagonist, the RANTES protein extended at the amino terminus by a single methionine residue was reported by Proudfoot, A. E. I., et al., J. Biol. Chem. (1996) 271:2599-2593. Elsner, J., et al., J. Biol. Chem. (2000) 275:7787-7794 showed that amino oxypentane—RANTES (AOP-RANTES) is a potent inhibitor of infection by human deficiency virus type I (HIV) strains. AOP-RANTES down regulates the CCR5 receptor but is less effective than RANTES itself in down modulation of CCR1. The ability of both RANTES and AOP-RANTES to inhibit HIV infection is also reported by Vila-Coro, A. J., et al., J. Immunol. (1999) 163:3037-3044. Gong, J -H, et al., J. Biol. Chem. (1996) 271:10521-10527 described RANTES antagonists lacking up to eight amino acids at the N-terminus. U.S. Pat Nos. 5,705,360; 5,739,103; 5,854,412; and 5,459,128 relate to N-terminal deletions of C-C chemokines, including RANTES. These N-terminal deleted chemokines are described as inhibiting endogenous chemokine binding and inhibiting activation of a responsive chemokine receptor.

PCT publication WO94/07512 assigned to the University of Texas System claims a method for treatment of allergic or chronic inflammatory disease which comprises administering the RANTES protein. PCT publication WO97/19696 to Lusso, P., et al., describes the use of RANTES and other C-C chemokines as having HIV suppressive activity; this publication claims a method of treating retrovirus infection by administering RANTES or related chemokines.

PCT publication WO98/13495 assigned to British Biotech Pharmaceuticals describes mutants of human RANTES which are said to retain the ability to inhibit HIV infection, but have reduced pro-inflammatory properties. These mutants are disaggregated and are modified forms of human RANTES where substitution mutations at Glu-26 and Glu-66 are apparently preferred. The construction of mutants with an alanine substituted for glutamic acid at position 26 and serine for glutamic acid at position 66 as well as double mutants with these substitutions are described. The applicants state that structural analysis indicates that substitution mutations at positions 1, 18, 23, 46, 52, 55, 60, 64 or 67 would also have the result of creating disaggregation mutants which have the properties of HIV inhibition/reduced pro-inflammation.

PCT publication WO99/11666 assigned to Gryphon Sciences describes N-terminally modified RANTES analogs, such as N-nonyl RANTES and AOP-RANTES and their use in treatment of allergic conditions and HIV infection.

PCT publication WO99/20759 to Genetics Institute is directed to N-terminally modified forms of a multiplicity of chemokines other than RANTES.

Recombinant viral vectors containing a RANTES-encoding insert are described and claimed as a method to inhibit binding of an immunodeficiency virus to cells in WO99/27122 assigned to TransGene S. A. WO99/28474 assigned to the U.S. government (DHHS) is directed to RANTES (3-68) and methods to inhibit HIV infection using this protein.

Construction of viral vectors containing RANTES encoding sequences are also disclosed by Braciak, T. A., et al., J. Immunol. (1996) 157:5076-5084 and by Youssef, S., et al., J. Clin. Invest. (2000) 106:361-367.

WO99/33989 to Fondazione Centro San Raffaele Del Monte Tabor claims RANTES mutants which are antagonists of the interaction between HIV virus and a chemokine receptor where there is a mutation in the N-terminal region, in the N loop region, in the 40's loop region or in all three regions.

WO99/37815 assigned to Akzo Nobel N. V. describes an isothermal transcription based amplification assay for detection or quantitation of chemokine RNA which is useful for the detection of levels of RANTES and related chemokines.

It has now been found that the RANTES chemokine has a significant effect on the respiratory infections caused by paramyxovirus. RANTES acts downstream of viral entry and signals through the specific CCR1 and/or CCR5 chemokine receptors as to interrupt the death pathway of macrophages which have been infected by the virus. RANTES not only inhibits apoptosis of the infected macrophage, but also clears the macrophages of infection.

### DISCLOSURE OF THE INVENTION

The invention is directed to methods of prophylactic and therapeutic treatment of paramyxovirus infection using RANTES protein or a recombinant system for RANTES expression. The invention also relates to a method to detect individuals who are susceptible to paramyxovirus infection by assessing the levels of expression of RANTES or its relevant receptors (CCR1 and CCR5) in such individuals or detecting defects in the genes for RANTES or its receptors. Knowledge of the interaction of RANTES with CCR1 and CCR5 permits these receptors to be used to identify alternatives to RANTES as medicaments in the treatment of paramyxovirus infection.

Thus, in one aspect, the invention is directed to a method to treat paramyxovirus infection which method comprises administering to a subject in need of such treatment an effective amount of RANTES protein or an effective amount of an expression system for said protein, including “naked DNA.” In another aspect, the invention is directed to a method of identifying individuals susceptible to paramyxovirus infection which method comprises assessing the level of expression of RANTES protein in said individuals, whereby an individual having an abnormally low level of such expression is identified as susceptible. Genetic testing can also be used to determine mutations in the genes encoding for RANTES and its receptors. The RANTES locus in humans has also recently been identified by Nickel, et al., J. Immunol. (2000) 164:1612-1616.

In additional aspects, the invention is directed to methods to identify medicaments which are useful in treating paramyxovirus infection by testing candidate medicaments for their ability to agonize CCR1 and/or CCR5 receptors. The invention is directed to medicaments so identified and to methods to treat paramyxovirus infection using these medicaments.

### BRIEF DESCRIPTION OF THE DRAWINGS

FIG. 1 is a cartoon which compares a model of the interaction of RANTES with CCR3 as related to HIV infection with the interaction of RANTES with CCR1 or CCR5 as related to paramyxovirus infection.

FIG. 2 shows the wild type RANTES genomic locus, a targeting construct to disrupt the first exon, and the resulting “null” locus.

FIG. 3 is a series of graphs comparing macrophage, PMN's, and lymphocytes at various times after infection in RANTES (+/+) and (−/−) mice.

FIG. 4 is a graph showing the survival of RANTES (+/+) compared to (−/−) mice after viral infection.

FIG. 5 is a series of graphs showing the effect of administering RANTES on viral load and cell death in infected mice.

### MODES OF CARRYING OUT THE INVENTION

The present invention centers on the discovery that the RANTES protein is able to prevent macrophage cell death and to enhance clearance of virus in infected subjects thus permitting the treatment of paramyxovirus infection using this protein or an expression system therefor. Because it is clear that RANTES protein is an endogenous defense with respect to paramyxovirus infection, it is also possible to screen populations for susceptibility to paramyxovirus infection by determining their status relative to endogenous production of effective RANTES protein. Thus, the present invention is directed to methods of treatment of paramyxovirus infection and methods to determine susceptibility to this infection. The latter is important as individuals identified as susceptible can be subjected to precautionary conditions and can be monitored for early treatment.

As used herein, “treat” or “treatment” refers both to therapeutic and prophylactic administration of the active ingredient. Thus, for example, susceptible individuals known to be in situations where they are exposed to paramyxovirus can be administered the RANTES protein or appropriate DNA or an individual already infected can be therapeutically administered these materials.

By “RANTES protein” is meant either the naturally occurring known human 68 amino acid protein or an analog thereof which retains the ability to activate the CCR1 and/or CCR5 receptor. As noted above, there are a number of analogs of the RANTES protein known in the art which behave as antagonists to the native RANTES protein. These analogs are believed to be ineffective in the method of the invention; rather analogs which retain the ability to activate the CCR1 and/or CCR5 receptor are included in the definition of “RANTES protein” herein. Among useful analogs are allelic variants which retain CCR5 activation activity and synthetically prepared modifications of the 68-mer. RANTES variants could be screened in a macrophage cell culture system or a transfected cell line that expresses the chemokine receptors CCR1 or CCR5. Receptor activation could be detected by calcium flux, chemotaxis, or apoptosis-inhibition assays. RANTES variants could be generated by targeted or random mutagenesis designed to generate compounds with receptor-activating properties.

The same assay systems are used to screen for small molecules other than peptides that selectively activate the RANTES signaling pathway for blocking apoptosis due to viral infection and for clearing the infection. A chemokine receptor antagonist has been identified with a similar approach (Liang, et al., J. Biol. Chem. (2000) 270:19000-19008). After candidates are identified, their therapeutic action may be verified in a mouse model of viral bronchitis.

Thus, a candidate medicament can be screened for its agonist ability with respect to the critical CCR1 and/or CCR5 receptor in a manner similar to that used above to confirm the agonist activity of analogs of the RANTES protein. Typically such medicaments are small molecules, although peptides could also be employed. By “small molecules” is meant the typical type of organic molecule generally found in pharmaceuticals and, in most cases, amenable to oral administration as indigestible in the digestive tract. Such small molecules are typically non-polymers, unlike peptides or polysaccharides.

The RANTES peptide or small molecule agonist may be used per se, or an expression system for the RANTES protein may be administered. By “expression system” is meant either a nucleotide sequence encoding the RANTES protein under control of additional sequences to effect its expression administered in a suitable vector or naked DNA—i.e., a nucleotide sequence encoding the RANTES protein administered in such a manner that delivery into the hosts' cells is able to effect expression.

As paramyxoviral infection is a respiratory infection, localized administration of the protein or the expression system for the encoding DNA is preferred. The dosage administered is designed to reproduce therapeutic levels of RANTES protein in the airway macrophages; the levels will be roughly equivalent to physiologic levels of 1 ng/ml in culture. Thus a preferred mode of administration is through an aerosol designed to obtain the appropriate levels in airway macrophage. Formulations for aerosols are well-known in the art and exemplary formulations may be found in Remington's Pharmaceutical Sciences, latest edition, Mack Publishing Co., Easton, Pa., the contents of which are incorporated by reference as they describe such formulations.

In a typical treatment, an aerosol formulation using standard excipients, and containing RANTES, RANTES variant, or small molecule RANTES agonist is administered through an inhaler. Typical protocols would employ 1 to 2 inhalations at a single dosage and the dosage would be adjusted to achieve maximal efficacy to prevent infection or to treat it after it has developed. Dosage would be determined by measurements of RANTES levels in cell culture and mouse model systems and by efficacy trials in these same model systems.

In addition to administering the small molecule medicament or a natural or engineered RANTES protein, a nucleotide sequence encoding said RANTES or RANTES variant protein, optionally coupled to control sequences for its expression may also be introduced into the subject. Typically, vectors for administration of nucleic acids include viral vectors such as adenovirus, retrovirus, Sendai virus, and the like. A wide variety of viral vector carriers, typically designed specifically for this purpose, are known in the art, and one study using a RANTES vector (Braciak, T. A., et al., J. Immunol. (1996) 157:5076-5084) and one using RANTES-encoding DNA vaccine (Youssef, S., et al., J. Clin. Invest. (2000) 106:361-367) have been reported.

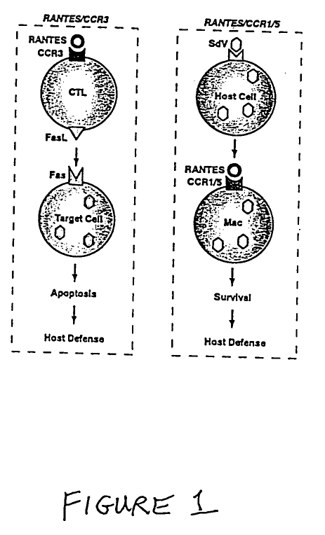

As shown hereinbelow, the effect of RANTES on paramyxoviral infection is different in mechanism from the known ability of RANTES protein or its antagonists to inhibit HIV infection. This is illustrated schematically in FIG. 1. In inhibiting HIV, as shown on the left, in addition to simply blocking the interaction of HIV with the CCR5 receptor, RANTES interacts with the CCR3 receptor on cytotoxic T lymphocytes to stimulate the production of FasL and induces consequent Fas-dependent killing of infected cells. In contrast, as shown in the right hand diagram, paramyxovirus stimulates the production of RANTES by infected host cells, such as airway epithelial cells and macrophage, which RANTES then binds to CCR1 and/or CCR5 on infected macrophage. The binding of RANTES to these infected macrophage inhibits apoptosis. The resulting viable macrophages then traffic normally out of the airway tissue and clear the infection.

In addition to providing means to treat paramyxoviral infection, the invention includes methods to detect individuals who are susceptible to paramyxoviral infection. In one embodiment, the ability of the individual to express the nucleotide sequence encoding the RANTES protein is determined by assessing levels of RANTES protein in, for example, airway epithelial cell and macrophage samples using standard detection techniques, for example, by ELISA or other immunoassay of bronchoalveolar or nasal lavage or serum obtained at the time of infection. Alternatively, the levels of RANTES encoding mRNA can be determined using, for example, kinetic PCR or the isothermal transcription based assay described in WO99/37815 noted above, and incorporated herein by reference using epithelial cells and macrophages obtained by airway lavage or using circulating leukocytes. In addition, the individual may be subjected to standard methods of genetic testing to determine the presence of mutations in the RANTES locus as identified recently (Nickel, et al., J. Immunol. (2000) 164:1612-1616).

The following examples are intended to illustrate but not to limit the invention.

### Preparation A

### Preparation of RANTES (−/−) Mice

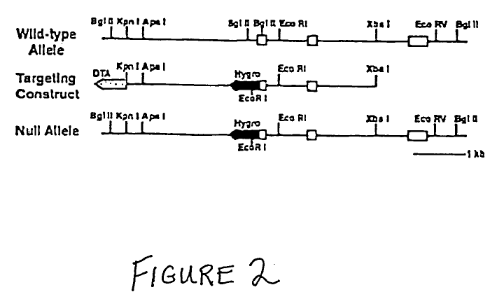

Mice containing a RANTES null locus were generated using a replacement vector derived from a 6.6 kb Apa I-Xba I fragment isolated from a lambda Fixil library prepared from mouse strain 129/Sv genomic DNA (Stratagene), as shown in FIG. 2. Exons are shown as open boxes. A334 Bgl II fragment spanning the transcription and translation start sites in exon 1 (−267 to 67) was replaced with a hygromycin-resistance cassette (Hygro from B. Jones, Princeton, N.J.) in opposite direction to the native gene. An upstream diphtheria toxin A cassette (DTA) was also added for negative selection. Linearized vector was transfected into E14 strain 129 embryonic stem cells, and after selection in hygromycin, targeted clones were injected into C57BL/6J blastocysts which were implanted to generate chimeric mice.

Chimeric males were mated with C57BL/6J female mice and transmission of RANTES null allele was verified by Southern blot analysis of tail DNA. RANTES deficiency was initially verified by Northern blot analysis and ELISA of peritoneal exudative cells stimulated with LPS. RANTES (±) mice from the third backcross into the C57BL/6J strain were used to generate (+/+) and (−/−) mice that were characterized by PCR of genomic tail DNA using primers (5′-GGGAACTTCCTGACTAGGGG-3′; 5′-CTGGACTGGAGGGCAGTTAG-3′; and 5′-AGTGAGGATGATGGTGAGGG-3′) corresponding to RANTES sequence in the null, wild-type, and both null and wild-type alleles, respectively.

Mice were maintained under pathogen-free conditions in the University biohazard barrier facility in micro-isolator cages for study at 7-9 weeks of age. Sentinel mice (specific pathogen-free ICN strain) and experimental control mice were handled identically to inoculated mice and exhibited no serologic or histologic evidence of exposure to 11 rodent pathogens (including Sendai virus (SdV)).

### EXAMPLE 1

### RANTES Expression in Infected Mice

Sendai virus (SdV, strain 52) and culture vehicle (allantoic fluid) were obtained from ATCC and stored at −70° C. After anesthesia, mice were inoculated intranasally with either 5,000 EID₅₀ (bronchitis) or 50,000 EID₅₀ (broncho-pneumonia) (50% egg infectious dose, EID₅₀) of SdV or with UV-inactivated SdV or culture vehicle diluted in 30 μL PBS.

The expression of RANTES was measured both by assessing lung tissue levels of RANTES mRNA and by immunostaining of lung tissue. Northern blot analysis used lung RNA isolated with RNA STAT-60 from TEL-TEST, Inc. (Friendswood, Tex.) and ³²P-labeled 0.5-kb mouse RANTES cDNA (EcoRI, XhoI fragment) from M. L. Shin, University of Maryland. In RANTES-positive mice, substantial amounts of mRNA were produced five days after infection at both 5,000 and 50,000 EID₅₀ doses; tracking of the 5,000 EID₅₀ dosed mice showed that the mRNA appeared after two days and appeared to reach a maximum at day 5, was less evident at day 8 and was not detectable at day 12. As expected, no RANTES mRNA was detected after five days post-infection in RANTES (−/−) mice infected with 50,000 EID₅₀.

Similar results were obtained using in situ hybridization with 35 S labeled RANTES cRNA and by immunostaining. For in situ hybridization, RANTES riboprobe was synthesized using the 0.5-kb RANTES cDNA fragment positionally cloned into the EcoRI and XhoI sites of pGEM3Zf-1 (Promega). Plasmid DNA was linearized either with ApaI or with BamHI and was transcribed in vitro with either T3 or T7 RNA polymerase to produce ³⁵S-UTP-labeled sense and antisense cRNA transcripts, respectively, using the Gemini Riboprobe system (Promega). For immunostaining trachea or lung at (25-cm water pressure) was fixed in 10% formalin, dehydrated in ethanol, embedded in paraffin, and cut into 5-μm thick sections. Tissue sections were treated with 10 mM Citra solution (Antigen Retrieval Citra; BioGenex, San Ramon, Calif.) for 15 min. at 98° C., blocked with 5% non-immune rabbit serum for 1 h at 25° C., and then incubated sequentially with goat anti-mouse RANTES Ab (5 μg/ml; R & D Systems, Minneapolis, Minn.) or nonimmune IgG for 24 h at 4° C., followed by biotinylated rabbit anti-goat IgG Ab (7.5 μg/ml) for 30 min at 25° C., streptavidin-conjugated alkaline phosphatase complex for 30 min at 25° C., and red chromogen (Vector Laboratories).

The pattern of RANTES induction appears the same as that for other epithelial immune response genes such as Stat1, ICAM-1 and IL-12 p40 (Walter, M. J., et al., (in press); Walter, M. J., et al., Am. J. Respir. Crit. Care Med. (2000) 161:A778. It is consistent with results in human airway epithelial cells infected with SdV or a different paramyxovirus, RSV. Koga, T., et al., Proc. Natl. Acad. Sci. U.S.A. (1999) 96:5680-5685.

### EXAMPLE 2

### Post-Infection Physiology

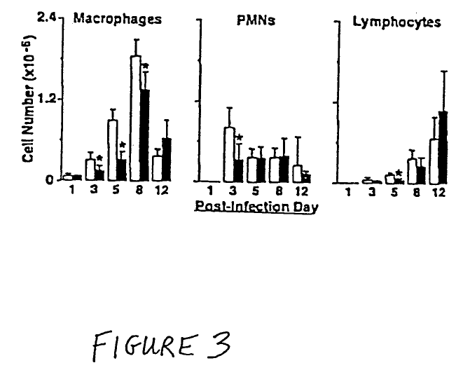

RANTES (+/+) and (−/−) mice were inoculated with 5,000 ED₅₀ SdV as described in Example 1 and BAL fluid was obtained by tracheal cannulation with 4 aliquots of 0.8 ml sterile PBS with 2% FBS. The BAL fluid was subjected to hopotonic lysis and centrifugation. The cell pellet was then used for determining total and differential cell counts as the mean of values from 3 blinded observers. The results are shown in FIG. 3, where the open bars represent (+/+) and the dark bars represent (−/−) mice. As seen, macrophage levels in both cases reach a maximum about 8 days after infection and lymphocytes increase through day 12. However, the (−/−) mice showed a decreased number of immune cells in the airspace as well as an accumulation of immune cells in the sub-epithelium.

In addition, lungs from post-infection days 3, 5, 8 and 12 were immunostained with anti-macrophage (Mac-3) monoclonal antibody and avidin-biotin horseradish peroxidase 3,3′-diaminobenzidine reporter and counterstained with hematoxylin the immunostaining showed that macrophage accumulated in the sub-epithelium at day 5, in the epithelium at day 8, and in the airspace by day 12 post-infection in the (−/−) mice.

In addition, when macrophage accumulated in the epithelium, RANTES (−/−) mice showed more virus in the epithelium than the (+/+) mice and were more lethargic, lost more weight, and had worse airway function.

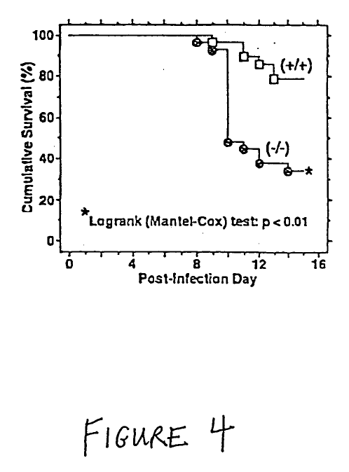

Using a similar protocol to Example 1, mice were inoculated at 50,000 EID₅₀. Serial lung sections were tested for viability by the TUNEL cell death assay which employs TdT-mediated dUTP Nicked End Labeling using the ApoTag Plus Fluorescein In Situ Apoptosis Detection kit from Intergen (Purchase, N.Y.). The results indicate enhanced survival of lung tissue cells in (+/+) mice compared to in (−/−) mice. In addition, at various times after infection, lungs were subjected to Western blotting against anti-SdV antibodies and detection by enhanced chemiluminescence. Bands corresponding to SdV hemagglutinin/neuraminidase and nucleocapsid proteins are present in both (+/+) and (−/−) mice after 5 days but disappear after 8 days. In addition, the mice were monitored for survival by Kaplan-Meier analysis (29 in each group); necropsy indicated bronchopneumonia. These results are shown in FIG. 4; the survival rate after 8-16 days in (+/+) mice is markedly higher than for (−/−) mice.

### EXAMPLE 3

### Effect of RANTES Treatment

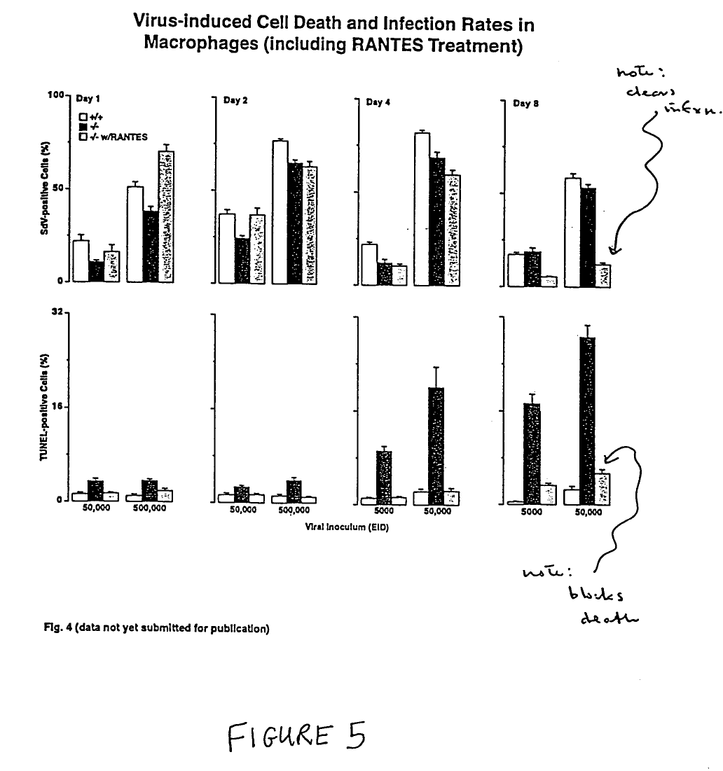

Macrophage cultures were prepared from RANTES (+/+) and (−/−) mice and incubated with 5,000 EID₅₀ or 50,000 EID₅₀ SdV for 4 days. The cultures were stained with anti-SdV antibody and CY3 reporter (red fluorescence) and counterstained with Hoescht dye (blue fluorescence) and then subjected to TUNEL reaction with FITC reporter (green fluorescence). In addition, a culture of macrophage from (−/−) mice was supplemented with 10 μg/ml RANTES (given transiently before infection). The results for these three groups are shown in FIG. 5. As shown, the percentage of SdV positive cells diminishes by day 8 in the cultures treated with RANTES and the presence of RANTES in the culture blocks cell death.

The top row of graphs in FIG. 5 shows that the level of infection after 8 days is roughly the same in both (+/+) and (−/−) mice; however, the addition of RANTES to the medium clearly shows that infection was cleared after 8 days. The clearing appears to commence even after day 4. The bottom row of graphs indicates that both RANTES (+/+) derived macrophage and macrophage treated with RANTES (10 μg/ml) resists apoptosis while macrophage derived from (−/−) mice and not treated with RANTES undergo substantial cell death even after day 4 and increasingly through day 8.

Initial rates of infection were not increased in RANTES deficiency, so the action of RANTES is distinct from that proposed at the level of the viral receptor, such as that described for HIV or cowpox virus. It appears RANTES acts via chemokine receptor mediated signaling via CCR5 to impact virus induction of mitochondrial permeability.

1. A method to treat paramyxovirus infection in a subject which method comprises administering to a subject in need of such treatment an amount of active ingredient which is RANTES protein or an expression system therefor effective to prevent or ameliorate paramyxovirus infection.

2. The method of claim 1 wherein said administering is via an aerosol formulation into the lungs.

3. The method of claim 1 wherein said effective amount is equivalent to 1 ng/ml of RANTES inf cell culture.

4. The method of claim 1 wherein said active ingredient is RANTES protein.

5. The method of claim 4 wherein said RANTES protein has the amino acid sequence of the mature protein in NCBI sequence accession No. NP002976.

6. The method of claim 1 wherein said active ingredient comprises a nucleotide sequence encoding RANTES protein.

7. A method to identify a subject at risk for paramyxovirus infection which method comprises assessing the level of expression of RANTES in said subject and comparing the level of expression with the expected level for normal population whereby a level of expression in said subject lower than the normal value identifies said subject as susceptible to paramyxovirus infection.

8. A method to identify a subject at risk for paramyxovirus infection which method comprises determining the presence or absence of a mutation in the RANTES gene of that said individual, whereby the presence of said mutation identifies said subject as susceptible to paramyxovirus infection.

9. A method to identify a medicament useful to treat paramyxovirus infection in a subject which method comprises
contacting cells that display the CCRI or CCR5 receptor with a candidate medicament; and
assessing the ability of said candidate medicament to agonize said CCR1 or CCR5 receptor;
whereby a candidate medicament that is able- to agonize the CCR1 or CCR5 receptor is identified as a medicament useful in treating paramyxovirus infection.

10. The method of claim 9 wherein said cells are macrophage.

11. The method of claim 9 wherein said assessing comprises measurement of calcium flux, chemotaxis, or apoptosis inhibition.

12. The method of claim 9 wherein the candidate medicament is an analog of RANTES obtainable by random mutagenesis.

13. The method of claim 9 wherein the candidate medicament is a small molecule.

14. A medicament identified by the method of claim 9.

15. A method to treat paramyxovirus infection in a subject which method comprises administering to a subject in need of such treatment an amount of an active ingredient which is the medicament of claim 14.
